(19) United States
(12) Patent Application Publication
(10) Pub. No.: US 2015/0355163 A1
(43) Pub. Date: Dec. 10, 2015

(54) T-TYPE CALCIUM CHANNEL INHIBITORS FOR TREATMENT OF CANCER

(71) Applicant: TAU THERAPEUTICS LLC, Charlottesville, VA (US)

(72) Inventors: Lloyd S. Gray, Louisa, VA (US); Timothy MacDonald, Charlottesville, VA (US)

(21) Appl. No.: 14/760,282

(22) PCT Filed: Jan. 10, 2014
(86) PCT No.: PCT/US14/11098

§ 371 Date: Jul. 10, 2015

Related U.S. Application Data
(60) Provisional application No. 61/751,038, filed on Jan. 10, 2013.

(51) Int. Cl. G01N 33/50; A61N 5/10; A61K 31/40; G01N 33/68; A61K 45/06; A61K 31/4184

(52) U.S. Cl. CPC G01N 33/502; A61K 45/06; A61K 31/4184; A61K 31/40; G01N 33/6872; A61N 5/10

(57) ABSTRACT

Presented herein are compounds that inhibit T-type Ca²⁺ channel activity in a cell when the cell membrane potential is about −90 mV. Preferred compounds inhibit T-type Ca²⁺ channel activity with an IC₅₀ of 10 μM or less at a membrane potential of about −90 mV. Preferred compounds show selectivity for inhibiting T-type Ca²⁺ channel activity at about −90 mV, relative to inhibition of T-type Ca²⁺ channel activity at about −30 mV to −60 mV, of 10:1 or less. Also provided are methods for identifying compounds that inhibit T-type Ca²⁺ channel activity in a cell when the cell membrane potential is about −90 mV, and compounds identified by such methods.

### CROSS-REFERENCE TO RELATED APPLICATIONS

This application claims the benefit of U.S. Provisional Application No. 61/751,038, filed Jan. 10, 2013, the entire disclosure of which is incorporated herein by reference.

### TECHNICAL FIELD

This disclosure relates to therapeutically useful compounds, methods of treatment, and methods to identify therapeutically useful compounds.

### BACKGROUND

Ca²⁺ influx at various points of the cell cycle is critical to progression, although the precise roles and pathways through which Ca²⁺ acts remain mostly elusive. Recently it has been possible to piece together one such pathway,^{1, 2} namely the role of Ca²⁺ influx in enabling passage through the G1/S transition or restriction point; a growth factor driven, unidirectional step in cell cycle progression. The G1/S transition serves to integrate information from a number of essential cellular inputs including growth factor signaling and nutrient availability. This restriction point is central to the cancer phenotype as genetic or epigenetic changes in a number of the key proteins in the G1 to S transition may allow cells to proliferate independently of mitogenic stimuli.³ Considerable effort has focused on targeting the cell cycle kinases to inhibit dysregulation of the G1/S and other transition points in the cell cycle.³ However, Ca²⁺ influx is a central element of the pathway for growth factor driven transition past the G1/S restriction point and no studies have been able to identify an acquired independence from this event—possibly because of the number of Ca²⁺ dependent processes that are integral to release from the restriction.

Calcium is a critical regulator of many cellular processes and, consequently, its influx is tightly controlled. In very general terms, this regulation can be either electrical or biochemical. Electrical control was the first of these regulatory mechanisms to be described and was outlined in the pioneering work of Hodgkin and Huxley (Huxley and Hodgkin, J. Physiol. 1:424-544 (1952). In this form of regulation, Ca²⁺ channels are opened to admit Ca²⁺ and subsequently closed in response to changes in the membrane potential. The details of this “gating” can be modified by biochemical events such as activation of protein kinase A⁴ or calmodulin,⁵ but the predominant regulatory event is alteration in the membrane potential, most notably in action potentials.

Intracellular calcium regulation is an important element of multiple signaling pathways regulating cell cycle transition and apoptosis. Cancer cells are able to progress through the cell cycle and bypass normal calcium-mediated checkpoints, indicating that cancer cells have developed alternative mechanisms to regulate intracellular calcium. New evidence that cancer cells express T-type calcium channels suggests that these channels play a role in checkpoint-independent cell cycle progression and cellular proliferation (Taylor J T et al., World J. Gastroenterol. 14:4984-4991, 2008).

The membrane potential is created by the presence of positively-charged ions in the intracellular space, such as sodium, potassium and calcium ions, at a concentration higher than the cell exterior. Membrane potentials in cells are typically in the range of −40 mV to −80 mV. In electrically excitable cells such as neurons, there are essentially two levels of membrane potential: the resting (non-excited) potential, and a higher, threshold potential. In a neuron, the resting potential is around −70 millivolts (mV) and the threshold potential is around −55 mV. Synaptic stimulation of a neuron causes the membrane potential to depolarize (rise) or hyperpolarize (fall). An action potential is a transitory “spike” in the electrical membrane potential of a cell. Action potentials are triggered when enough depolarization accumulates to bring the membrane potential up to threshold.

Although all cells have a membrane potential, most cells do not possess the molecular machinery or cellular geometry to generate action potentials. Nonetheless, all cells use increases in cytosolic Ca²⁺ to regulate processes such as secretion or cell division. These cells are thought to initiate Ca²⁺ influx by depletion of an internal Ca²⁺ storage depot in what is called capacitive Ca²⁺ entry.⁶ However, this mechanism may not be operative in the process of cell division and, if so, it would not be relevant to cancer biology or therapy.⁷ Complex models for the participation of components of the capacitive pathway have been introduced to implicate them in regulating the Ca²⁺ influx critically necessary for cell division,⁷ but a role for this pathway in cell division remains unclear. A number of ion channels have been suggested as the molecular pathway through which Ca²⁺ passes to enable the G1/S transition,⁸ although no consensus that a single pathway is predominant in a cell lineage, not just a cell line, has been achieved.

Evidence has accumulated that describes the regulation of Ca²⁺ channels in electrically excitable cells. There is also evidence that outlines the regulation of Ca²⁺ entry in electrically non-excitable cells, but this is unlikely to account for the entry of Ca²⁺ that is needed for cell division and transit past the G1/S boundary. Then, there are T-type Ca²⁺ channels that are expressed in cancer and stem cells, but which are voltage gated. Because most types of cancer cells and stem cells don't have action potentials that are thought necessary to regulate such voltage gated channels, there is little understanding of the function or regulation of these channels.

### SUMMARY

This disclosure provides compounds that inhibit T-type Ca²⁺ channel activity in a cell when the cell membrane potential is about −90 mV. Preferred compounds inhibit T-type Ca²⁺ channel activity with an IC₅₀ of 10 μM or less at a membrane potential of about −90 mV. Preferred compounds are also selective for inhibition of T-type Ca²⁺ channel activity at a membrane potential of about −90 mV, and show selectivity for inhibiting T-type Ca²⁺ channel activity at about −90 mV, relative to inhibition of T-type Ca²⁺ channel activity at about −30 to −60 mV, of 10:1 or less. Such compounds are useful for preventing cellular proliferation, and can prevent proliferation of cancer and other neoplastic cells while exhibiting little or no inhibition of neuronal activity.

This disclosure further provides methods for inhibiting the proliferation of cancer cells by administering an effective amount of a compound that inhibits T-type Ca²⁺ channel activity in a cell when the cell membrane potential is about −90 mV, as described above. The cancer cells can be any cancer cells, such as epithelial cancer cells or cancer stem cells. In certain embodiments, the compound administered is mibefradil or TH-1177.

This disclosure also provides methods for treating cancer in a subject by administering to a subject in need of cancer treatment an effective amount of a compound that inhibits T-type Ca²⁺ channel activity in a cell when the cell membrane potential is about −90 mV, as described above. The cancer can be any cancer, such as epithelial cancer. In certain embodiments, the compound administered is mibefradil or TH-1177. In a further embodiment, the subject is human.

Further disclosed herein are pharmaceutical compositions for the treatment of cancer, which contain at least the compounds disclosed herein.

This disclosure further provides methods of identifying compounds that inhibit T-type Ca²⁺ channel activity in a cell when the cell membrane potential is about −90 mV. These methods include determining the ability of a compound to inhibit T-type Ca²⁺ channel activity in a cell when the cell membrane potential is held at about −90 mV. The membrane potential can be held at about −90 mV by techniques known in the art, such as the patch-clamp technique. The ability of a compound to inhibit T-type Ca²⁺ channel activity in a cell when the cell membrane potential is about −90 mV can be determined, for example, by determining the ability of the compound to prevent growth factor-stimulated calcium entry into the cell. Calcium entry into the cell can be determined by measuring increases in levels of intracellular calcium, such as by use of a calcium sensitive fluorescent dye.

The present disclosure also provides a method for identifying a compound for utility in inhibiting cell cycle progression through the G1/S check point, inhibiting proliferation of cells in a cellular proliferative disorder, and/or enhancing the efficacy of radiation and/or a chemotherapeutic agent in treating a cellular proliferative disorder. The method includes determining that the compound inhibits T-type Ca²⁺ channel activity in a cell when a first cell membrane potential of the cell is held at a potential in the range from about −70 mV to about −110 mV; and, based on the determination, identifying a compound for utility in inhibiting cell cycle progression through the G1/S check point, inhibiting proliferation of cells in treating a cellular proliferative disorder, and/or enhancing the efficacy of radiation and/or a chemotherapeutic agent in treating a cellular proliferative disorder.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic representation of one of the pathways linking growth factor receptor activated Ca²⁺ with the biochemical cascade leading to transit past the G1/S restriction point.

FIG. 2 is a diagrammatic representation of the steps for growth factor-regulated activation of T-type Ca²⁺ channels. [Ca²⁺]_{I} is the intracellular Ca²⁺ concentration and ψ is the membrane potential.

### DETAILED DESCRIPTION

In the present disclosure it will be appreciated that that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

This disclosure provides treatments for cancer and neoplastic or proliferative diseases, involving inhibition of T-type Ca²⁺ channels. The inventors have determined that inhibition of T-type Ca²⁺ channel activity, specifically by inhibiting T-type Ca²⁺ channel activity in a cell when the cell membrane potential is about −90 mV, can prevent the progression of neoplastic disorders, and treat cancer.

The present invention is related to the discovery that inhibition of voltage-gated T-type Ca²⁺ channels by inhibition of responsiveness at specific membrane potentials is useful in the treatment of neoplastic or cancer cell proliferation. Unlike typical chemotherapeutic agents, antagonists that selectively inhibit T-type Ca²⁺ channel activity at membrane potentials about −90 mV can prevent proliferation of cancer cells, with limited or no effect on immune system function. Accordingly, administration of such antagonists is herein presented as a treatment for cancer.

Compounds that block T type calcium channels can exhibit either neuronal-like activity (which can be used in the treatment of pain, epilepsy, etc.), antiproliferative activity (which can be used in the treatment of cancer, etc.), or occasionally both activities. There are several possibilities to rationalize the differences in the behaviors of compounds that block T type calcium channels, such as potential differences in the channels (e.g., post-translational modifications) between the T-type channels in neurons and proliferating cells. Others have suggested that the activity of anti-proliferative compounds at T-type calcium channels is incidental and unrelated to the mechanism of anti-proliferation; that the anti-proliferative mechanism is a different target altogether.

The inventors have discovered that effective anti-proliferative compounds block T-type channels with IC₅₀ values less than about 10 mM when the cellular potential is held at −90 mV. Compounds that block calcium entry through T type calcium channels with high potency when the potential is −40 mV are effective in neuronal disorders. A compound demonstrating selectivity for anti-proliferative activity is preferably a compound with an IC₅₀ value at the −90 mV state, relative to the −40 mV state, of <10 (i.e., the IC₅₀ value at about −90 mV is 10 times or less the IC₅₀ value at −40 mV).

Mibefradil preferentially blocks the −90 mV state and is antiproliferative. TTL-1170 and chlopimozide, other anti-proliferative compounds with different scaffolds, are identified herein as showing similar selectivity. Other compounds that exhibit potent neuronal activity without anti-proliferative activity (e.g., TTA-A2 and MK-8998) show decreased selectivity at −90 mV relative to at −40 mV. Accordingly, this disclosure encompasses methods to identify compounds that inhibit T-type Ca²⁺ channel activity in a cell when the cell membrane potential is about −90 mV, as well as any compound identified through the use of this experimental protocol or its obvious extensions for anti-proliferative activity.

A “T-type calcium channel” or “T-type Ca²⁺ channel” is a low voltage activated ion channel with Ca²⁺ selective al subunits of the type of, or having similar activity and/or amino acid sequence identity to, Cav3.1 encoded by the CACNA1G gene, Cav3.2 encoded by the CACNA1H gene, or Cav3.3 encoded by the CACNA1I gene. In one embodiment, the T-type Ca²⁺ channel has the α1 subunit Cav3.2 encoded by the CACNA1H gene.

“Inhibition” as used herein refers to reduction or prevention of activity.

An “antagonist” or “inhibitor” inhibits activity or function. For example, a compound can act as an antagonist or inhibitor by inhibiting, reducing or eliminating protein expression, or preventing protein activity, or preventing interaction of protein with other proteins, resulting in an inhibition of a protein-mediated function or signaling. Examples of antagonist/inhibitor compounds include peptides, polypeptides, proteins, antibodies, antisense oligonucleotides, RNAi/siRNA, small molecules, chemotherapeutic agents, and fragments, derivatives and analogs thereof, that inhibit T-type Ca²⁺ channel activity. In one example, the compound inhibits T-type Ca²⁺ channel activity with a half maximal inhibitory concentration (IC₅₀) of less than about 10 μM when the cell membrane potential is about −90 mV. In another example, the selectivity of a compound for inhibiting T-type Ca²⁺ channel activity when the cell membrane potential is about −90 mV, relative to the selectivity of the compound for inhibiting T-type Ca²⁺ channel activity when the cell membrane potential is about −30 to −60 m V, is 1:10 or less.

Exemplary compounds of the invention inhibit T-type Ca²⁺ channel activity with a half maximal inhibitory concentration (IC₅₀) of less than about 10 μM when the cell membrane potential is about −90 mV. The IC₅₀ is a measure of the effectiveness of a compound in inhibiting biological activity. Methods to determine the IC₅₀ of a compound are known in the art and include functional antagonist assays, for example using a dose response curve, or competition binding assays that measure, for example, the ability of a compound to displace a known binding partner from a target molecule.

Activities of a T-type Ca²⁺ channel which can be inhibited by the present invention include, but are not limited to: cellular calcium uptake; regulation and/or mediation of intracellular calcium levels; regulation and/or mediation of intracellular window currents; calcium-mediated signaling and/or regulation of calcium signaling pathways; enabling passage through the G1/S transition or restriction point; enabling cell cycle progression; initiating and/or maintaining cellular growth and proliferation, particularly excessive or unwanted proliferation; initiating and/or maintaining neoplasia and/or tumor growth; and initiating and/or maintaining angiogenesis and/or metastasis.

The inventors have discovered that inhibition of T-type Ca²⁺ channel activity in a cell when the cell membrane potential is about −90 mV can preferentially inhibit unwanted cellular proliferation, such as cancer cell proliferation.

As used herein, the terms “about” and “approximately” indicate that a value includes the inherent variation based for example on the method being employed to determine the value, or naturally occurring variation, such as variation in resting or membrane potential found in a single cell, or variation in resting or membrane potential found between different cells. In one non-limiting embodiment the terms are defined to be within 10%, within 5%, within 1%, or within 0.5%. Similarly, a membrane potential of “about −90 mV” can include membrane potentials within a measured range of −80 mV to −100 mV, or within a range of −85 mV to −95 mV, or within a range of −89 mV to −91 mV. In another example, a membrane potential of “about −30 to −60 mV” can includes membrane potentials within a range of −20 mV to −70 mV, or within a range of −25 mV to −65 mV, and also encompasses membrane potential ranges such as about −30 mV to −40 mV, about −30 mV to −50 mV, about −30 mV to −70 mV, about −40 mV to −50 mV, about −40 mV to −60 mV, about −40 mV to −70 mV, about −50 mV to −60 mV, and about −50 to −70 mV, as well as about −30 mV, about −40 mV, about −50 mV, and about −60 mV.

The terms “selectivity” and “specificity” are used interchangeably herein to refer to the preference for inhibition at one state or condition over another state or condition. Selectivity or specificity can be absolute, indicating inhibition only at one state or condition and no inhibition at a different state or condition. Selectivity or specificity can also be relative, indicating some inhibition at one state or condition (i.e., for a cell or cell type at one membrane potential) and also some inhibition at another state or condition (i.e., for the same cell or cell type at a different membrane potential).

A compound demonstrating selectivity for anti-proliferative activity is exemplified as a compound with an IC₅₀ value at the −90 mV state, relative to about the −40 mV state, of 10:1 or less, i.e., the IC₅₀ value of a compound at a membrane potential of about −90 mV is no more than ten times the IC₅₀ value of the same compound at a membrane potential of −30 mV to −60 mV, or at about −40 mV. For example, the IC₅₀ of a compound such as mibefradil for inhibiting T-type Ca²⁺ channel activity at a cell membrane potential of −80 mV to −90 mV can be approximately 1 μM, while the IC₅₀ of a compound such as mibefradil for inhibiting T-type Ca²⁺ channel activity when the cell membrane potential is about −30 mV to −60 mV, can be about 0.1 μM or greater, such as 0.15 μM, 0.2 μM, 0.25 μM, 0.3 μM, up to 1.0 μM or greater.

Although the membrane potential of cells is about −30 mV in early G1 it falls to about −60 mV in late G1 then drops quickly to about −90 mV as the cell exits G1 and enters the S phase.¹ It is at this point that the T type calcium channel opens to allow the G1/S transition. Thus, T-type calcium channel blockers with high potency at inhibiting channels when they are at about −30 mV to −60 mV will have little effect on entry into S phase. Examples of such compounds are TTA-A2 and MK-8998 (see Kraus et al., J. Pharmacol. Exp. Ther. 335: 409-17 (2010) and U.S. Pat. No. 7,875,636). These compounds have high potency for inhibition of the T-type calcium channel, but have little or no effect on the proliferation of cancer cells. Thus, high potency blockade of T-type calcium channels per se does not predict clinical utility in the treatment of cancer.

The situation with TTA-A2 and MK-8998 is distinct from that of another T type calcium channel blocker, mibefradil. While mibefradil preferentially blocks channels at about −30 mV to −60 mV over −90 mV, this preference is about 10 to 1 [Gomora et al., J. Pharmacol. Exp. Ther. 292:96-103 (2000)] rather than about 1000 to 1 for other compounds [Kraus et al., J Pharmacol. Exp. Ther. 335: 409-17 (2010)]. This marked difference is reflected in the ability of mibefradil to inhibit cancer cell proliferation as shown in the Figures. This inhibitory action of mibefradil gives it the potential to have clinical utility in the cancer unlike the more potent blocker MK-8998.

Thus, the potency of a pharmaceutical agent to block T type channels per se does not confer clinical utility in the treatment of cancer. Rather, the ability to block T type calcium channels at about −90 mV is a critical attribute. Further, high potency binding at about −30 mV to −60 mV is irrelevant and may contribute to undesired effects of the pharmaceutical agent.

Accordingly, compounds that selectively inhibit T-type Ca²⁺ channel activity in a cell when the cell membrane potential is about −90 mV can inhibit unwanted cellular proliferation, while having little or no effect on neuronal activity relative to compounds such as TTA-A2 and MK-8998. In addition, compounds that selectively inhibit T-type Ca²⁺ channel activity in a cell when the cell membrane potential is about −90 mV can treat cancer cell proliferation, while having minimal effect on immune cell function relative to other chemotherapeutic compounds.

T-type Ca²⁺ channels are activated and inactivated by small membrane depolarizations, and display slow deactivation rates. Thus, these channels can carry depolarizing current at low membrane potentials and mediate cellular “window” currents, which occur within the voltage overlap between activation and steady state inactivation at low or resting membrane potentials (Tsien R W, et al. in Low-voltage-activated T-type Ca²⁺ channels, Chester: Adis International Ltd, pp. 1-394, 1998; Crunelli V, et al., J. Physiol. 562:121-129, 2005). T-type Ca²⁺ channels can maintain window current at non-stimulated or resting membrane potentials, thereby allowing a sustained inward calcium current carried by a portion of channels that are not inactivated (Bean B P, McDonough S I, Neuron 20:825-828, 1998). Mediation of window current allows T-type Ca²⁺ channels to regulate intracellular calcium levels, both in electrically firing cells such as neurons, and in non-excitable tissues, under non-stimulated or resting cellular conditions.

Like all voltage gated ion channels, T-type Ca²⁺ channels have three primary states, which are closed, opened and inactivated.²⁵ In simple terms, voltage gated channels cycle in a particular sequence: closed, open, inactivated; closed, open, inactivated; etc. As might be expected in voltage gated channels, these various states can be induced by experimentally imposed changes in membrane potential. In these experimental systems, T-type Ca²⁺ channels are mostly inactivated at the resting membrane potential of cancer cells (−60 mV) and are mostly closed, and available for opening, at the hyperpolarized potentials (about −90 mV) caused by activation of Ca²⁺ activated K⁺ channels.

The strongest evidence to date for a universal Ca²⁺ entry pathway enabling the G1/S transition has been presented for the voltage gated T-type Ca²⁺ in cells not derived from the marrow.^{2, 9, 10} Since the first description of T-type Ca²⁺ channels in cancer cells in 1992,¹¹ evidence for the physical and functional expression in cancer cells of T-type Ca²⁺ channels has mounted.¹² But the suggestion of a central role for voltage gated Ca²⁺ channels in cells that do not generate action potentials, such as cancer cells, has been met with skepticism.

The evidence for T-type Ca²⁺ channel involvement is derived from several lines of research. First, manipulation of T-type Ca²⁺ channels in cell lines by incorporation of interfering RNA targeting T-type Ca²⁺ channels blocks or slows proliferation of these cells by inhibiting transit past the G1/S boundary.^{13,14} Conversely, up regulation of T-type Ca²⁺ channel expression increases the rate of proliferation.¹⁵ In addition, pharmacologic inhibitors from disparate chemical classes inhibit T-type Ca²⁺ channels and concordantly block proliferation of cancer cells by inhibiting transit past the G1/S boundary.¹⁶ In addition, mRNA for the T-type Ca²⁺ channel isoform Cav3.2 (calcium channel, voltage-dependent, T-type, alpha 1H subunit) and/or its δ25 splice variant has been found in a variety of cancer cell types.^{16,17} Moreover there is a 1:1 concordance of the presence or absence of Cav3.2 message and drug sensitivity.¹⁷

T-type Ca²⁺ channels have “electrically-regulated” or “action potential-regulated” activity in that the channels open to admit calcium and close in response to changes in the membrane potential, particularly in response to alterations in action potentials across the membrane. For example, T-type Ca²⁺ channels are mostly inactivated at resting membrane potentials of about −30 mV to −60 mV, but become closed, and available for opening, either by calcium-activated calmodulin (CaM), or by a calmodulin activated protein such as CaMKII, at hyperpolarized potentials of about −90 mV.

T-type Ca²⁺ channels have “growth factor-regulated” activity in that the channels open to admit calcium following growth factor signaling. For example, activation of growth factor receptors by growth factors such as, but not limited to, insulin-like growth factor, epidermal growth factor, nerve growth factor, transforming growth factors and platelet derived growth factor, initiates a signaling cascade that changes T-type Ca²⁺ channels from inactivated to closed and available for opening. This mechanism can also be initiated by any agent, such as thapsigargin, that releases Ca²⁺ from an intracellular Ca²⁺ storage pool, such as the endoplasmic reticulum.

Accordingly, T-type Ca²⁺ channels are regulated by both electrically-regulated and growth factor-regulated mechanisms. For example, growth factor binding leads to changes in membrane potential that change T-type Ca²⁺ channels from inactivated to closed and available for opening, as in ER. The unique low voltage sensitivity of T-type Ca²⁺ channel states—clearly distinct from the high voltage activated L, N, P, R and Q type Ca²⁺ channels—is profiled exactly by the voltage regulation induced during growth factor induced proliferation. Thus, the resting state membrane potential and growth factor-mediated, activation-induced hyperpolarized potential during the G1/S transition of cancer and stem cells aligns precisely with the voltage-dependent states of T-type Ca²⁺ channels.

Exemplary compounds inhibiting T-type Ca²⁺ channel activity are disclosed in WO 00/059882, the contents of which are hereby incorporated by reference in their entirety.

In a particular embodiment, an inhibitor of T-type Ca²⁺ channel activity is TH-1177, with the formula as disclosed in WO 00/59882.

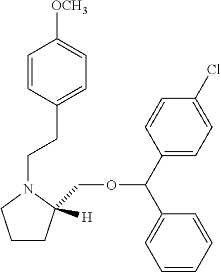 [SMILES: COc1ccc(CCN2CCC[C@H]2COC(c2ccccc2)c2ccc(Cl)cc2)cc1]

Examples of additional T-type Ca²⁺ channel activity inhibitors include, but are not limited to, mibefradil, bepridil, clentiazem, diltiazem, fendiline, gallopamil, prenylamine, semotiadil, terodiline, verapamil, amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, and perhexyline. In a preferred example, the growth factor-regulated T-type Ca²⁺ channel activity inhibitor is mibefradil or TH-1177.

Compounds such as mibefradil or TH-1177 inhibit T-type Ca²⁺ channel activity when the cell membrane potential is about −90 mV. Similarly, agents that bind to the site occupied by mibefradil or TH-1177 can inhibit T-type Ca²⁺ channel activity in a cell when the cell membrane potential is about −90 mV.

This disclosure further provides methods of identifying compounds that inhibit T-type Ca²⁺ channel activity in a cell when the cell membrane potential is about −90 mV. Such compounds can be identified by measuring inhibition of T-type Ca²⁺ channel activity in a cell using standard electrophysiological methods such as patch clamp or by measuring the ability of a pharmaceutical agent to block calcium entry into a cell, such as a cancer cell, when that cell is stimulated by a mitogen, such as a growth factor. Such methods are disclosed, for example, in Densmore, et al., FEBS Lett. 312:161-164 (1992); Haverstick, et al., Mol. Biol. Cell 4:173-184 (1993); and Gomora et al., J. Pharmacol. Exp. Ther. 292:96-103 (2000), the contents of which are incorporated by reference. Calcium entry can be determined by methods such as intracellular entrapment of a Ca²⁺ sensitive fluorescent dye.

Accordingly, this disclosure encompasses methods to identify compounds with antiproliferative activity and/or ability to treat cancer, by determining the ability of a compound to inhibit T-type Ca²⁺ channel activity in a cell when the cell membrane potential is about −90 m V. This disclosure further encompasses compounds identified by the methods disclosed herein.

As used herein, a “neoplastic” cell or “cancer” cell means an abnormal cell exhibiting uncontrolled proliferation and potential to invade surrounding tissues.

As used herein, the term “cancer stem cell” refers to a cell that can be a progenitor of, or give rise to a progenitor of, a highly proliferative cancer cell. A cancer stem cell has the ability to re-grow a tumor as demonstrated by its ability to form tumors in immuno-compromised mammals such as mice, and to form tumors upon subsequent serial transplantation in immuno-compromised mammals such as mice.

The compounds disclosed herein can inhibit proliferation, differentiation or development of neoplastic or cancer cells. Cancer or a neoplastic disease, including, but not limited to, neoplasms, tumors, metastases, leukemias or any disease or disorder characterized by uncontrolled cell growth, can be prevented, treated, and/or managed by administering to a subject in need thereof a therapeutically effective amount of an inhibitor of T-type Ca²⁺ channel activity as disclosed herein.

Any type of cancer can be prevented, treated and/or managed in accordance with the invention. Non-limiting examples of cancers that can be prevented, treated and/or managed in accordance with the invention include cancers of epithelial origin such as breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

The methods of treatment and compositions provided herein are further useful for inhibiting proliferation of stem cells such as cancer stem cells.

The vital role of T-type Ca²⁺ channels in the G1/S transition is not limited to cancer cell proliferation. Embryonic stem cells also contain message for Cav3.2 that increases at the G1/S transition, pharmacologic inhibitors of Cav3.2 block proliferation of them and interfering RNA directed at Cav3.2 decreases alkaline phosphatase and Oct 3/4 expression, which characterize early stem cells.¹⁸ Taken at face value, these data show that the expression of Cav3.2 is critical for cell cycle progression in stem cells. The data for embryonic stem cells additionally suggest that T-type Ca²⁺ channel levels are involved in maintaining their undifferentiated state.¹⁷ However it has also been shown that homozygous Cav3.2 knockout mice develop normally displaying only abnormal coronary artery function and significantly lower birthweight.¹⁸

Taken together, it is apparent that the function of Cav3.2, normally necessary for cell cycle progression and embryonic cell self-renewal, can be taken over by another Ca²⁺ influx mechanism in its absence. Given the regulatory and biophysical similarities among the three T-type Ca²⁺ isoforms (Cav3.1, 3.2 and 3.3), it is reasonable to speculate that the normal function of Cav3.2 can be subserved by one of the two other isoforms. Known pharmacologic T-type Ca²⁺ antagonists do not significantly differentiate among the three isoforms¹⁹ and this could explain the inability of cancer cells grown in the continuous presence of a T-type Ca²⁺ blocker (at its 1C₃₀) for as long as year to develop resistance to the same drug (D. M. Haverstick, University of Virginia, unpublished observations).

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

As used herein, “treatment” refers to clinical intervention in an attempt to alter the disease course of the individual or cell being treated, and can be performed either for prophylaxis or during the course of clinical pathology. Therapeutic effects of treatment include without limitation, preventing occurrence or recurrence of disease, alleviation of symptoms, diminishment of any direct or indirect pathological consequences of the disease, decreasing the rate of disease progression, amelioration or palliation of the disease state, and remission or improved prognosis. For example, treatment of a cancer patient may be reduction of tumor size, elimination or reduction of neoplastic or malignant cells, prevention of metastasis, or the prevention of relapse in a patient whose tumor has regressed.

As used herein, the terms “therapeutically effective amount” and “effective amount” are used interchangeably to refer to an amount of a composition of the invention that is sufficient to result in the prevention of the development, recurrence, or onset of cancer stem cells or cancer and one or more symptoms thereof, to enhance or improve the prophylactic effect(s) of another therapy, reduce the severity and duration of cancer, ameliorate one or more symptoms of cancer, prevent the advancement of cancer, cause regression of cancer, and/or enhance or improve the therapeutic effect(s) of additional anticancer treatment(s).

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

For example, an effective amount of an inhibitor of T-type Ca²⁺ channel activity, may be between 0.0001 to 10 mg/kg of body weight daily. The dosage range will generally be about 0.5 mg to 1.0 g. per patient per day which may be administered in single or multiple doses. In one embodiment, the dosage range will be about 0.5 mg to 200 mg per patient per day; in another embodiment about 1 mg to 100 mg per patient per day; and in another embodiment about 1 mg to 50 mg per patient per day; in yet another embodiment about 10 mg to 20 mg per patient-per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising about 1 mg, 2 mg, 3 mg, 4 mg, 10 mg, 100 mg, 200 mg or 250 mg active ingredient. The compounds may be administered on a regimen of 1 to 4 times per day, such as once or twice per day.

In certain embodiments of the invention, the therapeutically effective amount is an amount that is effective to achieve one, two or three or more of the following results once it is administered: (1) a reduction or elimination of the neoplastic cell population; (2) a reduction or elimination in the cancer cell population; (3) a reduction in the growth or proliferation of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) the size of the tumor is maintained and does not increase or increases by less than 10%, or less than 5%, or less than 4%, or less than 2%, (10) an increase in the number of patients in remission, (11) an increase in the length or duration of remission, (12) a decrease in the recurrence rate of cancer, (13) an increase in the time to recurrence of cancer, (14) an amelioration of cancer-related symptoms and/or quality of life and (15) a reduction in drug resistance of the cancer cells.

In some embodiments, the amount or regimen of an inhibitor of electrically regulated T-type Ca²⁺ channel activity results in a reduction in the bulk tumor size as well as a reduction in the cancer stem cell population. In certain embodiments, the reduction in the bulk tumor size; the reduction in the bulk tumor size and the reduction in the cancer stem cell population, including drug resistant cancer stem cells; or the reduction in the bulk tumor size, the reduction in the cancer stem cell population and the reduction in the cancer cell population are monitored periodically. Accordingly, in one example, the invention provides a method of preventing, treating and/or managing cancer in a subject, the method comprising: (a) administering to a subject in need thereof one or more doses of an effective amount of an inhibitor of electrically-regulated T-type Ca²⁺ channel activity. In a particular example, the inhibitor inhibits CACNA1H.

The terms “proliferation” and “growth” as used interchangeably herein with reference to cells, refer to an increase in the number of cells of the same type by cell division, rapid and repeated cellular reproduction, cell cycling, and cell growth, particularly uncontrolled cellular growth. “Development” refers to the progression from a smaller, less complex, or benign form to a larger, more complex, or neoplastic form. For example, a tumor may develop from a small mass to a larger mass. Cancer stem cell development can refer to the progression from a non-cancerous cell state to a cancerous cell state, or the progression from non-neoplastic tissue formation to neoplastic or tumor formation.

A “cellular proliferative disorder” means a disorder wherein cells are made by the body at an atypically accelerated rate. A cellular proliferative disorder can include cancer. Non-limiting examples of cancers include bladder cancer, brain cancer, breast cancer, colorectal cancer, cervical cancer, gastrointestinal cancer, genitourinary cancer, head and neck cancer, lung cancer, ovarian cancer, prostate cancer, renal cancer, skin cancer and testicular cancer.

More particularly, cancers that may be treated by the compound, compositions and methods described herein include, but are not limited to, the following: (1) Breast cancers, including, e.g., ER⁺ breast cancer, ER⁻ breast cancer, HER2⁻ breast cancer, HER2⁺ breast cancer, stromal tumors such as fibroadenomas, phyllodes tumors and sarcomas and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, luminal B, basal A, basal B, and triple negative breast cancer, which is estrogen receptor negative (ER⁻), progesterone receptor negative, and HER2 negative (HER2⁻). In some embodiments, the breast cancer may have a high risk Oncotype score; (2) cardiac cancers, including, e.g., sarcoma, e.g., angiosarcoma, fibrosarcoma, rhabdomyosarcoma, and liposarcoma; myxoma; rhabdomyoma; fibroma; lipoma and teratoma; (3) Lung cancers, including, e.g., bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma; (4) Gastrointestinal cancer, including, e.g., cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma; (5) Genitourinary tract cancers, including, e.g., cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma; (6) Liver cancers, including, e.g., hepatoma, e.g., hepatocellular carcinoma; cholangiocarcinoma; hepatoblastoma; angiosarcoma; hepatocellular adenoma; and hemangioma; (7) Bone cancers, including, e.g., osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; (8) Nervous system cancers, including, e.g., cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma; (9) Gynecological cancers, including, e.g., cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma; (10) Hematologic cancers, including, e.g., cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenström's macroglobulinemia; (11) Skin cancers, including, e.g., malignant melanoma, basal cell carcinoma, squamous cell carcinoma, Kaposi's sarcoma, moles dysplastic nevi, lipoma, angioma, dermatofibroma, keloids, and psoriasis; (12) Adrenal gland cancers, including, e.g., neuroblastoma; (13) Pancreatic cancers, including, e.g., exocrine pancreatic cancers such as adenocarcinomas (M8140/3), adenosquamous carcinomas, signet ring cell carcinomas, hepatoid carcinomas, colloid carcinomas, undifferentiated carcinomas, and undifferentiated carcinomas with osteoclast-like giant cells; and exocrine pancreatic tumors.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A cellular proliferative disorder can also include non-cancerous proliferative disorders including, but not limited to, hemangiomatosis in newborns, secondary progressive multiple sclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenoisis, and cirrhosis.

The term “chemotherapeutic agent” as used herein refers to an agent that can be used to kill or inhibit the growth or proliferation of cells in the treatment of a cellular proliferative disorder. Examples of suitable chemotherapeutic agents include any of: abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Biochemical Activation of T-Type Ca²⁺ Channels Driving G1/S Transition.

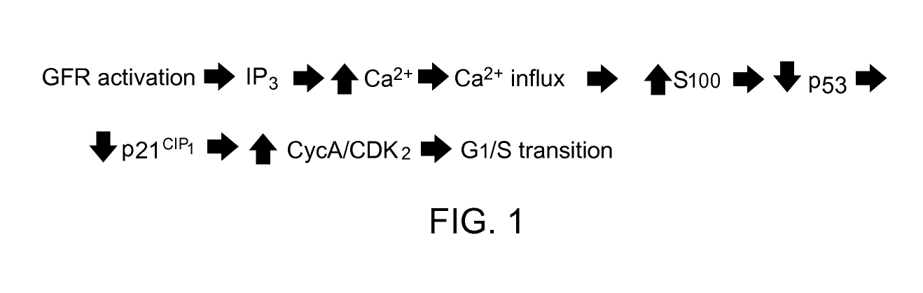

This disclosure proposes the following sequence of steps from initial growth factor activation to release of the G1/S restriction, as illustrated in FIG. 1. Growth factor receptor (GFR) activation increases the cytosolic inositol trisphosphate (IP3) concentration through activation of phospholipase C. IP3 then releases Ca²⁺ from the internal storage pool through interaction with the IP3 receptor on the endoplasmic reticulum. The resulting small increase in the cytosolic Ca²⁺ concentration triggers a much larger increase resulting from Ca²⁺ influx through T-type Ca²⁺ channels, as outlined in FIG. 1. A necessary event in the pathway involves Ca²⁺ binding S100, which in tum binds to and inactivates p53, thus relieving activation of p21. Because activated p21 inactivates CDK2, reduction in p21 activity allows CDK2 to drive the G1/S transition.

Events Leading to Cell Division in Electrically Non-Excitable Cells.

A model has been presented for the events that follow growth factor receptor activation leading to cell division. In this model, the Ca²⁺ released from its internal depot activates Ca²⁺ entry by clearly Ca²⁺ dependent process rather than Ca²⁺ entry being triggered secondarily by the “emptiness” of the internal depot.¹⁷ Simply, Ca²⁺ released from the storage depot activates calmodulin, which in turn activates the Ca²⁺ influx leading to cell division.

The membrane potential of cancer cells has been reported to be between −30 mV to −60 mV. However when membrane potential was measured as a function of position in the cell cycle in a human breast cancer line, it was shown to be about −30 mV in early G1 falling to about −60 mV in late G1 and S (Ouadid-Ahidouch et al., Am. J. Physiol. Cell. Physiol., 287:C125-34 (2004)), which may account for the variability of measured membrane potential in cancer cells reported in the literature. Growth factor activation produces inositol triphosphate, which releases Ca²⁺ from an internal storage depot.²⁰ One of the first actions of this increase in intracellular Ca²⁺ can be the activation, and opening, of Ca²⁺ activated K+ channels.²¹ The resulting efflux of K⁺ will naturally result in a transient decrease in the membrane potential from the value of about −60 mV in late G1 to a hyperpolarized value of about −90 mV, the equilibrium potential for potassium.

Interestingly, K⁺ channel blockers have been shown to inhibit growth factor stimulated increases in cytosolic Ca²⁺ and to block cellular proliferation by inhibiting transit past the G1/S boundary in cancer cell lines and mesenchymal stem cells,²²⁻²⁴ an action functionally identical to T-type Ca²⁺ channel inhibitors. While the K⁺ channel blockers used in such studies are promiscuous, it is unexpected that a K⁺ channel, or the hyperpolarization associated with K⁺ channel activity, would have an effect on Ca²⁺ channel function or would increase cytosolic Ca²⁺, leading to cell division. A widely cited belief is that the hyperpolarization mediated by K⁺ channel function serves to increase the electrochemical driving force for Ca²⁺ entry. On the face of it, this is clearly true. However, there is a 10,000-fold concentration gradient for Ca²⁺ entry at a membrane potential of 0 and it is difficult to reconcile the metabolic burden required to hyperpolarize the plasma membrane potential and the need to have tightly controlled Ca²⁺ entry with the generally hypothesized role of hyperpolarization in increasing the driving force for Ca²⁺ entry. Accordingly, activation of K⁺ channels and the attendant drop in membrane potential toward potassium's equilibrium potential is herein disclosed as functioning to increase the driving force for Ca²⁺.

According to a controversial but nonetheless popular hypothesis, a malignant tumor is comprised of a variable proportion of so-called cancer stem cells (Lathia J D et al., Stem Cell Rev. 7:227-37 (2011)). These cells are reported to be relatively resistant to radiation and chemotherapy and could account for cancer recurrence. Cancer stem cells are thought to be similar to embryonic stem cells and knowledge of the biology of both types of stem cells may reveal novel therapeutic strategies. Interestingly, Cav3.2 (Unigene cluster Hs.459642) and the type 2 small conductance calcium activated potassium channel (Unigene Cluster Hs.98280) have strikingly similar early gestational co-expression patterns as determined by the National Center for Biotechnology Information with the highest expression in the embryoid body falling off thereafter. This early gestational expression pattern is not seen with Cav3.1 or Cav3.3 nor is it seen with other calcium activated potassium channels. This co-expression pattern is consistent with the functional expression of Cav3.2 in embryonic stem cells¹⁸ as well as the model described below, and may help to reveal new medical approaches to cancer treatment.

A Model for Growth Factor Regulated Ca²⁺ Influx Enabling Proliferation.

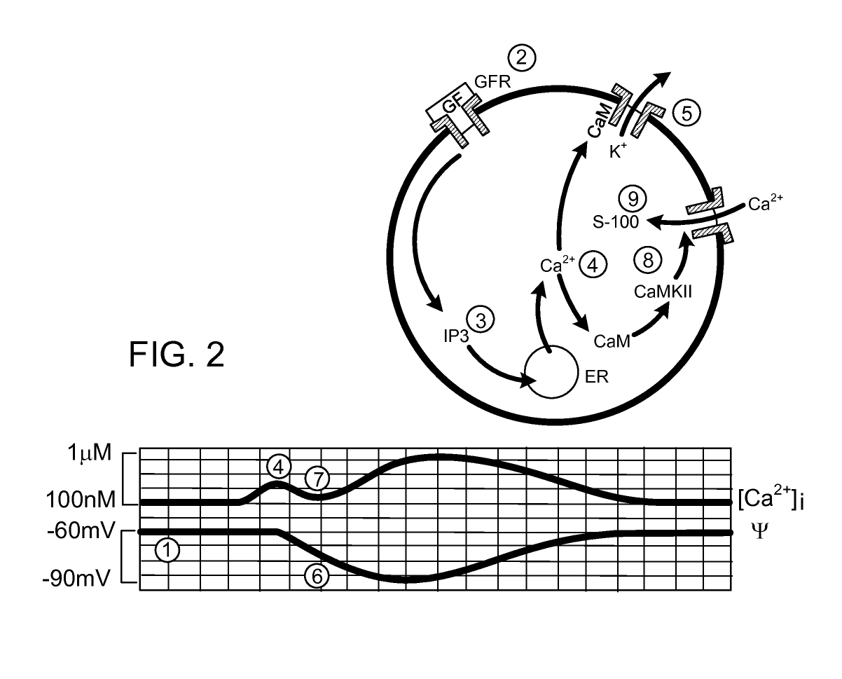

These observations can be synthesized into a coherent and simple model (FIG. 2):
1. At the resting membrane potential, T-type channels are inactivated and unable to be opened.
2. Growth factor receptor is activated.
3. This causes the production of inositol trisphosphate.
4. Inositol triphosphate releases Ca²⁺ from an internal storage pool.
5. This released Ca²⁺ opens Ca²⁺ activated K+ channels via constitutively bound calmodulin.
6. The resulting hyperpolarization relieves inactivation of T-type channels.
7. T-type channels are now closed and, thus, available to be opened.
8. Ca²⁺ activated calmodulin diffuses to and opens T-type channel perhaps via T-type channel phosphorylation by a calmodulin kinase.
9. A Ca²⁺ activated S100 isoform inactivates p53 removing activation of p21, which releases CDK2 to propel progression into S phase.

These steps are further described as follows. In the first arm of the pathway, the constitutive association of CaM with Ca²⁺ activated K+ channels^{5,25} allows for rapid opening of them in response to an increase in cytosolic Ca²⁺. The need for diffusion of the Ca²⁺/CaM complex and the possible requirement for the participation of CaMKII will slow the second arm of the pathway, possibly providing the temporal sequencing of hyperpolarization followed by CaM dependent activation of Ca²⁺ entry via T-type Ca²⁺ channels.

Among the various points at which this pathway can be attacked for therapeutic gain, a vulnerable target is the T-type Ca²⁺ channel itself. One reason for this vulnerability is the limited number of T-type Ca²⁺ channel isoforms. Growth factors, for example, consist of a large number of related proteins that can be recruited to bypass one that has been blocked. There are only three T-type Ca²⁺ channel proteins and all are about equally sensitive to available pharmacologic inhibitors¹⁹ so that recruitment of an alternative member would be futile.

Another point of vulnerability results from the restricted distribution of this protein, which is normally expressed in embryonic stem cells, and not expressed in cells that do not normally divide in adults, but that is re-expressed in response to injury or carcinogenic stimulus. This re-emergent proliferation can result from something as relatively simple as re-expression in fibroblasts dividing in response to wound healing,²⁶ which is a standard response to a pathological stimulus, or as complex as in solid cancers, which may well be a pathologic response to a normal stimulus. In addition, bone marrow derived cells appear to utilize a different Ca²⁺ entry pathway, as T-type channel antagonists have no effects on proliferation or differentiation of these cells and no expression of Cav3.2 is observed in cell lines derived from bone marrow. The molecular basis for this is not understood, but is the source of active research. These attributes makes inhibitors of T-type Ca²⁺ channels very appealing candidates for a new and unique category of cancer chemotherapeutic agents that inhibit proliferation of cancer cells while having reduced or no effect on immune cell proliferation.

As monotherapy, T-type calcium channel blockers slow cancer cell proliferation and reduce tumor growth in vivo as observed in a number of animal models of human disease.^{27, 28} Mibefradil is a T-type Ca²⁺ channel blocker that was marketed by Roche for the treatment of hypertension and angina (Clozel et al., Cardiovasc. Drug Rev. 9:4-17 (1991)). It was withdrawn from the market after being used by almost a million patients when it was discovered to have undesirable drug-drug interactions caused by mibefradil's inhibition of CYP 450 3A4 (Po and Zhang, Lancet. 351:1829-30 (1998)). Aside from this, mibefradil was remarkably well tolerated and devoid of side effect even for a member of its therapeutic class (Kobrin et al., Am. J. Cardiol. 80:40C-46C (1997)). This suggests that side effects of T-type Ca²⁺ channel blockers will be modest at most and significantly better than those generally caused by many cancer chemotherapy drugs. In part because of this, use of T-type Ca²⁺ channel blockade—as a cell cycle and cancer stem cell targeted cytostatic agent—is actively being pursued.

However, there is another possibility for the potential clinical utility of such agents. Most conventional cytotoxic agents act at a particular stage of the cell cycle, usually during DNA synthesis. If cancer cells could be “lined up” at the G1/S restriction point and then released into S phase, conventional cytotoxins might be made more efficient at killing cancer cells. This appears to be the case in a murine model of human glioblastoma (Keir et al., J. Neurooncol. 111(2):97-102 (2013)). In this model, mice were treated with a seven day course of mibefradil to block Ca²⁺ influx and halt progression through the cell cycle at the G1/S restriction point, then 30 minutes after the last dose of mibefradil a five day course of temozolomide was started. This regimen significantly increased the cytotoxic effect of temozolomide and restored the sensitivity to temozolomide of drug resistant cancer cell lines. An IND using this strategy in glioblastome multiforme opened in early 2012, a phase 1 study of escalating doses of mibefradil in normal, healthy volunteers is underway, and a trial in patients was initiated by the National Cancer Institute (NCI)'s Adult Brain Tumor Consortium in the Spring of 2012. Further details of the method are provided in WO 2010/141842, which is incorporated herein by reference.

In some embodiments, the present disclosure provides a method for identifying a compound for utility in inhibiting cell cycle progression through the G1/S check point, inhibiting proliferation of cells in a cellular proliferative disorder, and/or enhancing the efficacy of radiation and/or a chemotherapeutic agent in treating a cellular proliferative disorder. The method includes determining that the compound inhibits T-type Ca²⁺ channel activity in a cell when a first cell membrane potential of the cell is held at a potential in the range about −70 mV to about −110 mV; and, based on the determination, identifying a compound for utility in inhibiting cell cycle progression through the G1/S check point, inhibiting proliferation of cells in treating a cellular proliferative disorder, and/or enhancing the efficacy of radiation and/or a chemotherapeutic agent in treating a cellular proliferative disorder. In some embodiments, the membrane potential can include can include membrane potentials within a measured range of about −80 mV to about −100 mV, or within a range of about 85 mV to about −95 mV, or within a range of about −89 mV to about −91 mV. In some embodiments, the membrane potential is about 90 mV. In some embodiments, the cells can express one or more of the T-type calcium channel sub-types described herein. In some embodiments, the cells can be engineered to recombinantly express one or more of the type calcium channel sub-types described herein.

In some embodiments, the method can include determining a first IC₅₀ that is the IC₅₀ of the compound in inhibiting the T-type calcium channel activity when a cell is held at the first cell membrane potential. The compound can be identified as useful for the utility based on a determination that the first IC₅₀ is about 10000 μM or less, about 1000 μM or less, about 1000 μM or less, about 100 μM or less, about 10 μM or less, about 1 μM or less, or about 100 nM or less.

In some embodiments, the method can include determining a second IC₅₀ of the compound, wherein the second IC₅₀ is the IC₅₀ of the compound in inhibiting the T-type calcium channel activity in a cell when the cell is held at a second cell membrane potential in the range from about −30 mV to about −60 mV. The second membrane potential is greater (i.e., less negative) than the first membrane potential. In various embodiments, the second membrane potential can be within a range from about −20 mV to about −70 mV, from about −25 mV to about −65 mV, from about −30 mV to about −40 mV, from about −30 mV to about −50 mV, from about −30 mV to about −70 mV, from about −40 mV to about −50 mV, from about −40 mV to about −60 mV, from about −40 mV to about −70 mV, from about −50 mV to about −60 mV, from about −50 to about −70 mV, as well as about −30 mV, about −40 mV, about −50 mV, or about −60 mV.

In some embodiments, the measurements at different membrane potentials are performed using the same cell or group of cells. In some embodiments, the measurements at different membrane potentials are performed using the different cells or group of cells. The cells used are preferably of the same cell type. For example the cells can be clones, cells from the same cell line or proliferating cells from a single subject in need of treatment for a cellular proliferative disorder.

In some embodiments, the method can include identifying a compound as being useful for the utility based on the determination that the ratio of the first IC₅₀ to the second IC₅₀ is about 20:1 or less, about 10:1 or less, about 5:1 or less, about 2:1 or less, about 1:1 or less, about 1:2 or less, about 1:5 or less, about 1:10 or less, or about 1:100 or less. The method can also include identifying a compound as having reduced or low liability for neuronally-mediated side-effects base on the determination that the ratio of the first IC₅₀ to the second IC₅₀ is about 20:1 or less, about 10:1 or less, about 5:1 or less, about 2:1 or less, about 1:1 or less, about 1:2 or less, about 1:5 or less, about 1:10 or less, or about 1:100 or less. Examples of neuronally based side-effects can include anxiety, attentive deficits, cognitive deficits, confusion, convulsions, depression, dizziness, hallucinations, psychosis, sedation, stimulation, etc.

In some embodiments, the cell membrane potential can be controlled using a patch-clamp technique. In some embodiments cell membrane potential can be controlled using any other technique described herein or known in the art.

In some embodiments, the ability of a compound to inhibit T-type Ca²⁺ channel activity is determined by determining the ability of the compound to inhibit growth factor-stimulated calcium entry into the cell. In some embodiments, the ability of a compound to inhibit T-type Ca²⁺ channel activity is determined using any other technique described herein or known in the art.

In some embodiments, calcium entry into the cell is determined by measuring increases in the levels of intracellular calcium using a calcium sensitive marker such as a calcium-sensitive fluorescent dye. In some embodiments calcium entry into the cell is determined by using any other technique described herein or known in the art.

In some embodiments, the method includes identifying the compound for utility in inhibiting cell cycle progression through the G1/S check point.

In some embodiments, the method includes identifying the compound for utility in inhibiting proliferation of cells in a cellular proliferative disorder. The cellular proliferative disorder can be a cancerous or non-cancerous proliferative disorder, including any one or more of the cancerous or non-cancerous proliferative disorders identified herein. The cellular proliferative disorder can be a disorder, the proliferating cells of which express T-type calcium channels. The cellular proliferative disorder can be a disorder, the proliferating cells of which express any isoform of a T-type calcium channels as described herein.

In some embodiments, the method includes identifying the compound for enhancing the efficacy of radiation and/or a chemotherapeutic agent in treating a cellular proliferative disorder when the compound is administered prior to administration of the radiation and/or chemotherapeutic agent. The cellular proliferative disorder can be a cancerous or non-cancerous proliferative disorder, including any one or more of the cancerous or non-cancerous proliferative disorders identified herein. The cellular proliferative disorder can be a disorder, the proliferating cells of which express T-type calcium channels. The chemotherapeutic agent can be any of the chemotherapeutic agents identified herein, or any combination thereof.

In some embodiments, the method can be performed wherein the cells used comprise one or more proliferating cells of a subject in need of treatment for the proliferative disorder and can identify the compound as being useful for the treatment of the cellular proliferative disorder and/or for use in enhancing the efficacy of radiation and/or a chemotherapeutic agent in treating a cellular proliferative disorder. In some embodiments, the compound is administered prior to administration of the radiation and/or chemotherapeutic agent. The method can be used to identify the compound as being useful for treatment of the subject. The cellular proliferative disorder can be a cancerous or non-cancerous proliferative disorder, including any one or more of the cancerous or non-cancerous proliferative disorders identified herein. The cellular proliferative disorder can be a disorder, the proliferating cells of which express T-type calcium channels. The chemotherapeutic agent can be any of the chemotherapeutic agents identified herein, or any combination thereof.

In some embodiments, the method includes administering to the subject an effective amount of the compound to the subject to treat the cellular proliferative disorder. In some embodiments, the method includes administering to the subject an effective amount of the compound in combination with an effective amount of radiation and/or the chemotherapeutic agent to the subject to treat the cellular proliferative disorder. In some embodiments, the compound is administered to the subject prior to administration of the radiation and/or chemotherapeutic agent. The cellular proliferative disorder can be a cancerous or non-cancerous proliferative disorder, including any one or more of the cancerous or non-cancerous proliferative disorders identified herein. The cellular proliferative disorder can be a disorder, the proliferating cells of which express T-type calcium channels. The chemotherapeutic agent can be any of the chemotherapeutic agents identified herein, or any combination thereof.

In some embodiments, the chemotherapeutic agent is selected from the group consisting of consisting of temozolomide, 5-fluorouracil, 6-mercaptopurine, bleomycin, carboplatin, cisplatin, dacarbazine, doxorubicin, epirubicin, etoposide, gemcitabine, hydroxyurea, ifosfamide, irinotecan, topotecan, methotrexate, mitoxantrone, oxaliplatin, paclitaxel, docetaxel, vinblastine, vincristine, vinorelbine; vindesine and mitomycin C. In some embodiments, the chemotherapeutic agent is temozolomide. In some embodiments, the chemotherapeutic agent is carboplatin. In some embodiments, the chemotherapeutic agent is gemcitabine.

In some embodiments, the cancer is selected from the group consisting of selected from the group consisting of brain cancer, breast cancer, colon cancer, glioma, glioblastoma, melanoma, ovarian cancer and pancreatic cancer. In some embodiments, the cancer is brain cancer. In some embodiments, the cancer is glioma. In some embodiments, the cancer is ovarian cancer. In some embodiments, the cancer is pancreatic cancer.

The invention has been described with reference to various embodiments and techniques. However, it should be understood that many variations and modifications can be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all sub-ranges and individual values are encompassed. This invention is not to be limited by the embodiments disclosed, including any exemplified in the specification, which are given by way of example or illustration and not of limitation. The scope of the invention shall be limited only by the claims.

All references cited herein are hereby incorporated by reference in their entirety.

### REFERENCES

1. Rustandi R R, Baldisseri D M, Weber D J. Structure of the negative regulatory domain of p53 bound to S100B (betabeta). Nat. Struct. Biol., 2000; 7: 570-4.
2. Lu F, Chen H, Zhou C, et al. T-type Ca²⁺ channel expression in human esophageal carcinomas: a functional role in proliferation. Cell Calcium, 2008; 43: 49-58.
3. Lapenna S, Giordano A. Cell cycle kinases as therapeutic targets for cancer. Nat. Rev. Drug Discov. 2009; 8:547-66.
4. Trautwein W, Hescheler J. Regulation of cardiac L-type calcium channels by phosphorylation and G proteins. Annu. Rev. Physiol., 1990; 52: 257-74.
5. Saimi Y, Kung C. Ion channel regulation by calmodulin binding, FEBS Lett., 1994; 350: 155-8.
6. Putney J W, Jr. A model for receptor-regulated calcium entry. Cell Calcium, 1986; 7: 1-12.
7. Smyth J T, Putney J W. Regulation of store-operated calcium entry during cell division. Biochem. Soc. Trans., 2012; 40: 119-23.
8. Cahalan M D. STIMulating store-operated Ca(2+) entry. Nat. Cell. Biol., 2009; 11: 669-77.
9. Harper T V, McLatchie L, Perez-Reyes E, Cribbs L L, Shattock M J, Brooks G. T-type calcium channel expression is necessary for G I-S progression in vascular smooth muscle. Circulation 2000; 102: 11-48.
10. Li W, Zhang S L, Wang N, Zhang B B, Li M. Blockade of T-Type Ca(2+) channels inhibits human ovarian cancer cell proliferation. Cancer Invest., 2011, 29(5): 339-46.
11. Densmore J J, Szabo G, Gray L S. A voltage-gated calcium channel is linked to the antigen receptor in Jurkat T lymphocytes. FEBS Lett. 1992; 312: 161-4.
12. Santoni G, Santoni M, Nabissi M. Functional role of T-type calcium channels in tumour growth and progression: Prospective in cancer therapy. Br. J. Pharmacol., 2012, 166(4): 1244-6.
13. Mulgrew C J, Cove-Smith A, McLatchie L M, Brooks G, Shattock M J, Hendry B M. Inhibition of human mesangial cell proliferation by targeting T-type calcium channels. Nephron Exp. Nephrol., 2009; 113: e77-88.
14. Rodman D M, Reese K, Harral J, et al. Low-voltage-activated (T-type) calcium channels control proliferation of human pulmonary artery myocytes. Circ. Res., 2005; 96: 864-72.
15. Brooks G, Harper J V, Bates S E, et al. Over expression of the voltage-gated T-type calcium channel induces vascular smooth muscle cell proliferation. Circulation, 1999; 100: 1-209.
16. Taylor J T, Zeng X B, Pottle J E, et al. Calcium signaling and T-type calcium channels in cancer cell cycling. World J. Gastroenterol., 2008; 14: 4984-91.
17. Gray L S, Perez-Reyes E, Gamorra J C, et al. The role of voltage gated T-type Ca²⁺ channel isoforms in mediating “capacitative” C;+ entry in cancer cells. Cell Calcium, 2004; 36: 489-97.
18. Rodriguez-Gomez J A, Levitsky K L, Lopez-Bameo J. T-type Ca²⁺ channels in mouse embryonic stem cells: modulation during cell cycle and contribution to self renewal. Am. J. Physiol. Cell. Physiol., 2012; 302: C494-504.
19. Heady T N, Gomora J C, Macdonald T L, Perez-Reyes E. Molecular pharmacology of T-type Ca²⁺ channels. Jpn. J. Pharmacol., 2001; 85: 339-50.
20. Exton J H. Regulation of phosphoinositide phospholipases by hormones, neurotransmitters, and other agonists linked to G proteins. Annu. Rev. Pharmacol. Toxicol., 1996; 36: 481-509.
21. Wonderlin W F, Strobl J S. Potassium channels, proliferation and G1 progression. J. Membr. Biol., 1996; 154: 91-107.
22. Chandy, K C, Wulff, H, Beeton C, Pennington M, Gutman G A, Cahalan M D. K(+) channels as targets for specific immunomodulation. Trends Pharmacol. Sci., 2004; 25: 280-9.
23. Strobl J S, Wonderlin W F, Flynn D C. Mitogenic signal transduction in human breast cancer cells. Gen. Pharmacol., 1995; 26: 1643-9.
24. Tao R, Lau C P, Tse H F, Li G R. Regulation of cell proliferation by intermediate conductance Ca²⁺-activated potassium and volume-sensitive chloride channels in mouse mesenchymal stem cells. Am. J. Physiol. Cell. Physiol., 2008; 295:CI409-16.
25. Levitan I B. It is calmodulin after all! Mediator of the calcium modulation of multiple ion channels. Neuron 1999; 22: 645-8.
26. Estacion M, Mordan L J. Expression of voltage-gated calcium channels correlates with PDGF-stimulated calcium influx and depends upon cell density in C3H 10T1I2 mouse fibroblasts. Cell Calcium, 1993; 14: 161-71.
27. Haverstick D M, Heady T N, Macdonald T L, Gray L S. Inhibition of human prostate cancer proliferation in vitro and in a mouse model by a compound synthesized to block Ca²⁺ entry. Cancer Res. 2000; 60: 1002-8.
28. Panner A, Wurster R D. T-type calcium channels and tumor proliferation. Cell Calcium 2006; 40: 253-9.
29. Giles T D. Hypertension and pathologic cardiovascular remodeling: a potential therapeutic role for T-type calcium antagonists. Clin. Ther., 1997; 19 Suppl. A: 27-38.

1. A method for identifying a compound for utility in inhibiting cell cycle progression through the G1/S check point, inhibiting proliferation of cells in a cellular proliferative disorder, and/or enhancing the efficacy of radiation and/or a chemotherapeutic agent in treating a cellular proliferative disorder, the method comprising:
determining that the compound inhibits T-type Ca²⁺ channel activity in a cell when a first cell membrane potential of the cell is held at a potential in the range from about −70 mV to about −110 mV; and
based on the determination, identifying a compound for utility in inhibiting cell cycle progression through the G1/S check point, inhibiting proliferation of cells in a cellular proliferative disorder, and/or enhancing the efficacy of radiation and/or a chemotherapeutic agent in treating a cellular proliferative disorder.

2. (canceled)

3. The method of claim 1, wherein the first cell membrane potential of the cell is held at a potential of about −90 mV.

4. The method of claim 1, further comprising determining a first IC₅₀ that is the IC₅₀ of the compound in inhibiting the T-type calcium channel activity when a cell is held at the first cell membrane potential.

5. The method of any claim 4, wherein identifying the compound for the utility is based on a determination that the first IC₅₀ is about 1000 μM or less.

6. The method of any claim 4, wherein identifying the compound for the utility is based on a determination that the first IC₅₀ is about 10 μM or less.

7. The method of claim 4, further comprising determining a second IC₅₀ of the compound, wherein the second IC₅₀ is the IC₅₀ of the compound in inhibiting the T-type calcium channel activity in a cell when the cell is held at a second cell membrane potential in the range from about −30 mV to about −60 mV.

8. (canceled)

9. The method of claim 7, wherein the second cell membrane potential is about −40 mV.

10. The method of claim 7, further comprising identifying a compound for the utility, or identifying that the compound has reduced liability for neuronally-mediated side-effects, based on the determination that the ratio of the first IC₅₀ to the second IC₅₀ is about 20:1 or less.

11. The method of claim 7, further comprising identifying a compound for the utility, or identifying that the compound has reduced liability for neuronally-mediated side-effects, based on the determination that the ratio of the first IC₅₀ to the second IC₅₀ is about 1:1 or less.

12-17. (canceled)

18. The method of claim 1, comprising identifying the compound for utility in inhibiting cell cycle progression through the G1/S check point.

19. The method of claim 1, comprising identifying the compound for utility in inhibiting proliferation of cells in a cellular proliferative disorder.

20. The method of claim 19, wherein the method is performed using one or more proliferating cells of a subject in need of treatment for the cellular proliferative disorder.

21. The method of claim 20, further comprising administering to the subject an effective amount of the compound to the subject to treat the cellular proliferative disorder.

22. The method of claim 1, comprising identifying the compound for utility in enhancing the efficacy of radiation and/or a chemotherapeutic agent in treating a cellular proliferative disorder.

23. (canceled)

24. The method of claim 22, wherein the method is performed using one or more proliferating cells of a subject in need of treatment for the cellular proliferative disorder.

25. The method of claim 24, further comprising administering to the subject an effective amount of the compound in combination with an effective amount of radiation and/or the chemotherapeutic agent to the subject to treat the cellular proliferative disorder.

26-27. (canceled)

28. The method of claim 22, wherein the chemotherapeutic agent is selected from the group consisting of temozolomide, 5-fluorouracil, 6-mercaptopurine, bleomycin, carboplatin, cisplatin, dacarbazine, doxorubicin, epirubicin, etoposide, gemcitabine, hydroxyurea, ifosfamide, irinotecan, topotecan, methotrexate, mitoxantrone, oxaliplatin, paclitaxel, docetaxel, vinblastine, vincristine, vinorelbine; vindesine and mitomycin C.

29-31. (canceled)

32. The method of claim 1, wherein the cellular proliferative disorder is a cancer.

33-43. (canceled)

44. A method for identifying a compound that inhibits T-type Ca²⁺ channel activity in a cell at a cell membrane potential of about −90 mV, comprising determining the ability of a compound to inhibit T-type Ca²⁺ channel activity in a cell when the cell membrane potential is held at about −90 mV.

45. (canceled)

46. The method of claim 44, wherein the ability of a compound to inhibit T-type Ca²⁺ channel activity in a cell at a cell membrane potential of about −90 mV is determined by determining the ability of the compound to prevent growth factor-stimulated calcium entry into the cell at said membrane potential.

47-48. (canceled)

49. A compound identified by the method of claim 44.

50-57. (canceled)
